(19) United States
(12) Patent Application Publication
(10) Pub. No.: US 2006/0228318 A1
(43) Pub. Date: Oct. 12, 2006

(54) Elastomer-forming barrier preparation

(76) Inventor: Tomas Fabo, Molnlycke (SE)

Correspondence Address: YOUNG & THOMPSON, 745 SOUTH 23RD STREET, 2ND FLOOR, ARLINGTON, VA, 22202, US

(21) Appl. No.: 10/553,953

(22) PCT Filed: Jun. 2, 2004
(86) PCT No.: PCT/SE04/00848

§ 371 Date: Jun. 12, 2006

(30) Foreign Application Priority Data
Jun. 10, 2003 (SE) 030167603

(51) Int. Cl. A61K 8/89

(52) U.S. Cl. 424/70.12

(57) ABSTRACT

A method and preparation for application to the skin (stratum corneum). The preparation includes a silicone composition which is highly viscous on application and which, after application, cures, by crosslinking, into a soft and skin-friendly elastomer which adheres to the skin.

### TECHNICAL FIELD

The present invention relates to a preparation for application to skin, to a method for applying a protective layer to skin and to a device for storing and applying such a preparation.

### BACKGROUND TO THE INVENTION

One of the most important functions possessed by human skin is to constitute the body's barrier to the environment. The skin protects against the harmful effects of microorganisms, toxic substances, heat, cold, mechanical damage, etc. The skin also constitutes a necessary protection against dehydration. The skin around wounds, in particular what are termed chronic wounds, which take anything from weeks to months to heal, is frequently in a worse condition than the remaining skin and its barrier function is consequently impaired. There can be several reasons for this circumstance. Some wounds originate in the main from underlying diseases which give rise to locally impaired circulation and nutrition, thereby weakening the skin and making it more easily damaged. Many wounds are moist and produce secretions which frequently leak out onto the skin around the wound, with the moisture causing the skin to disintegrate. The wound secretion contains enzymes, microorganisms and other substances which can have a harmful effect on the skin, particularly on disintegrating skin. Dressings which are used on wounds are frequently provided with self-adhesive glue. The purpose of the self-adhesive glue is for it to adhere to the skin around the wound and fix the dressing at the intended site. However, a disadvantage of self-adhesive glue is that the skin can be damaged when the dressing is removed and that disagreeable pain can simultaneously ensue. It is especially troublesome when the self-adhesive dressings have to be changed frequently over a relatively long period. Systemic or local treatment with cortisone, radiation, cell poisons or other medical preparations can weaken the skin still further.

The most frequently employed method of protecting the skin around wounds is to lubricate skin with a protective ointment, cream or paste. Ordinary paraffin, e.g. Vaseline, is sometimes employed. Other frequently occurring preparations, which are often termed “barrier creams” in English usage, are ACO zinc paste (ACO hud AB (ACO Skin AB], Upplands Väsby, Sweden), Baza® Protect from Coloplast (Coloplast Corp. Marietta, Ga., USA), 3M™ Durable Barrier Cream (3M Health Care, St. Paul, Minn., USA), Inotyol (Laboratoires URGO, Dijon, France) and Silon (Smith & Nephew AB, Mölndal, Sweden). A barrier cream increases the resistance of the skin to liquid and other harmful substances which come to be on the skin. The protective ointment/cream/paste layer prevents the wound liquid from coming into direct contact with the skin. When weeping wounds are bandaged, use is frequently made of a relatively thick, approx. 1 cm-wide, layer of a highly viscous ointment or paste (for example zinc paste) on the skin closest to the wound. A thinner layer of a moisture-preserving, protective ointment or cream is applied outside this strand in order to prevent the skin from drying out and thereby improve the intrinsic barrier function of the skin.

When ointments/creams/pastes are used around wounds, they also have a function in addition to protecting the covered skin. The ointment/cream/paste prevents peripheral leakage of wound liquid from the wound to the skin outside the wound at the same time as it protects against passage of liquids, for example urine, from the outside and inside the wound.

Many preparations comprise active components such as hydrocortisone, urea, ZnO and antimicrobial substances which reduce the irritation of the skin and/or facilitate healing of the skin. The ointment or cream is sometimes applied by means of what is termed an ointment compress, which is a more or less sparse textile material which is impregnated with an ointment of the abovementioned type. The compress is laid over the region of the wound such that it extends for some distance over the skin.

In the present application, the terms ointment, cream, paste and highly viscous are defined as described below.

An ointment consists of an anhydrous ointment base which is composed of a mixture of oil, fat and wax as well as any added substances which give the ointment its specific properties. The added substances can, for example, consist of pharmaceutical preparations, herbal extracts, cosmetics, dyes, vitamins, enzymes, etc. Ointments contain either no added water or only very small quantities of added water.

A cream is an emulsion of water in an ointment base or ointment base in water. Creams can also contain added quantities of different fat-soluble and/or water-soluble substances.

According to a common definition, a paste is an ointment which contains more than 40% solid substances.

Ointments, creams and pastes have a consistency which is such that they can readily be spread on skin using fingers or a hand.

The abovementioned ointments, creams and pastes suffer from several disadvantages. They possess very low cohesion and are therefore felt to be sticky and are frequently difficult to keep in place under a dressing since they do not have any dimensional stability but instead behave as viscous liquids. They can leak into the wound, be absorbed in the wound dressing or leak out from the region of the wound and soil clothes, etc. Self-adhesive dressings cannot be fixed to skin which is coated with these preparations since the adhesion is inactivated. As a result, leakage of wound liquid between the skin and the dressing frequently occurs. Wiping off old paste and ointment is frequently a time-consuming step when changing dressings.

Another method is to protect the skin by applying, to the skin around a wound, a liquid which contains a solid substance which is dissolved or dispersed in a volatile liquid. U.S. Pat. No. 5,741,509 provides an example of such a method. Following application to the skin, the volatile substance evaporates off and leaves behind a protective film of the solid substance. The liquid can be applied in spray form or by being spread with a cotton wad, for example. An example of this type of product is 3M™ Cavilon™ No Sting Barrier Film (3M Health Care, St. Paul, Minn., USA).

A disadvantage of this method is that it is difficult to remove the protective film from the skin and it is also difficult to obtain a sufficiently thick layer of the protective material; as a result, the method is not always sufficiently effective. Nor does this type of product adequately prevent peripheral leakage of wound liquid from the wound or passage of urine, etc., into the wound from the exterior.

Skin-protecting ointments/pastes also have an important function in situations other than those connected with caring for the skin around wounds. For example, sensitive and damaged skin is also found around different types of stomata and where the skin is penetrated with different types of tubes. Leakage of more or less aggressive body liquids frequently occurs in connection with these applications and the skin is subjected to frequent changes of self-adhesive dressings. While ointments are often used in this connection, they can be problematical to employ when they prevent adhesion of stoma bags or other articles which may need to be attached.

The object of the invention is to provide a preparation which is easy to apply to the skin and which produces a protective layer on the skin which does not suffer from the disadvantages of the abovementioned preparations.

### SUMMARY OF THE INVENTION

This object is achieved by means of a preparation for application to the skin (stratum corneum), characterized in that it comprises a silicone composition which is highly viscous on application and which, after application to the skin, cures, by means of crosslinking, to form a soft and skin-friendly elastomer which adheres to the skin.

In this patent specification, “highly viscous” is understood as meaning a liquid, ointment, paste or cream whose viscosity is between 5-300 Pa·s at a shearing rate of 10 s⁻¹. Preparations having a viscosity of more than 300 Pa·s do not function in this application since it then becomes difficult to spread them on the skin.

According to a preferred embodiment, the preparation has, on application, a viscosity of 5-300 Pa·s, preferably 10-120 Pa·s, more preferably 20-80 Pa·s, and, after curing, a penetration of 2-15 mm, preferably 3-10 mm. After having cured on the skin the preparation expediently has an adherence to the skin of 0.3-3.0 N/25 mm. The curing time following application is 0.5 min-24 hrs, preferably 1 min-1 hr, more preferably 1-5 min.

The silicone composition in the preparation preferably consists of an addition-curing RTV silicone system. The crosslinkable substance in the silicone system can consist of polydimethylsiloxane, with some of its methyl groups being replaced with vinyl groups, and the crosslinking-forming substance can consist of dimethylsiloxane with some of its methyl groups being replaced with hydrogen, and the composition contains a platinum-based catalyst.

One or more skin care substances can have been added to the silicone composition.

The invention also relates to a method for affixing a protective layer to the skin, characterized in that a preparation comprising a silicone composition, which is highly viscous on application and which, following application to the skin, cures, as a result of crosslinking, to form a soft and skin-friendly elastomer which adheres to the skin, is applied to the skin, after which the preparation is allowed to cure, as a result of crosslinking, to form a soft, skin-friendly elastomer which adheres to the skin.

In a preferred embodiment, the preparation is applied at a layer thickness of 0.1-5 mm.

An article for medical use, such as a stoma bag, a tube, parts of a wound dressing or a bandage, can be applied to the upper side of the preparation, i.e. the side which faces away from the skin, before the preparation has cured. In a variant, the preparation is applied to the article for medical use before it is applied to the skin in conjunction with the article.

The preparation can advantageously be designed such that its adherence to the article for medical use is greater than the adherence of the preparation to the skin after curing, resulting in the preparation accompanying the article when the latter is removed.

The preparation can be applied around a wound, directly outside the edge of the wound, with a breadth of 2-100 mm.

In an advantageous implementation of the method, one or more wound dressings can be applied such that the dressing or dressings cover(s) the wound and the area to which the preparation has been applied, with the dressing or dressings being applied before the preparation has cured. The wound dressing or wound dressings are preferably liquid-tight dressings. If dressing systems consisting of several different layers are used, it is sufficient for one of the layers to be liquid-tight.

### LIST OF FIGURES

The invention will now be described with reference to the attached figures, of which;

FIG. 1 shows, in diagrammatic form, a perspective view of a wound surrounded by a preparation in accordance with a preferred embodiment of the invention,

FIG. 2 shows, in diagrammatic form, a cross sectional view of a wound surrounded by a preparation in accordance with a preferred embodiment of the invention in interaction with an overlying dressing, and

FIGS. 3 and 4 show, in diagrammatic form, a cross sectional view of a protective layer according to the invention to which a stoma bag is attached.

### DESCRIPTION OF EMBODIMENTS

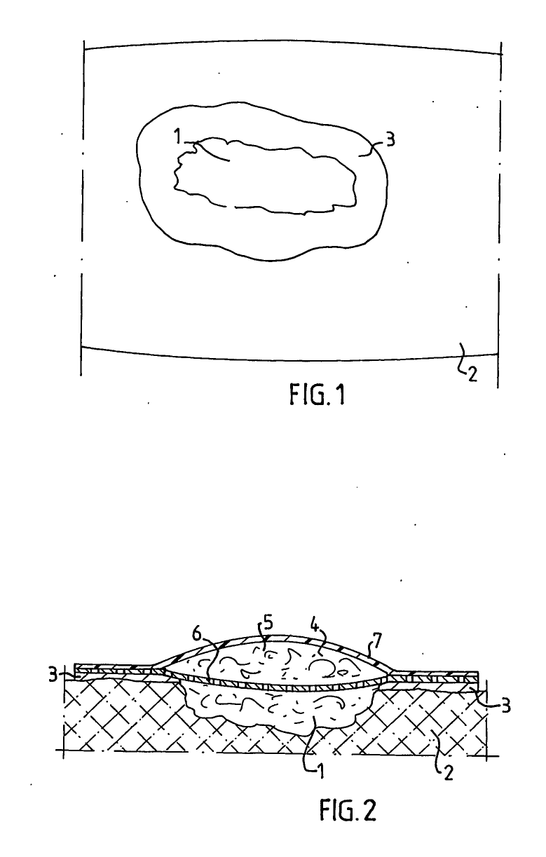

FIG. 1 shows, in diagrammatic form, a wound 1 on, for example, an arm 2. A preparation in accordance with the present invention has been applied, in a layer 3 which is 0.1-5 mm thick, around the wound. The preparation layer 3 has an ointment-like consistency on application and the preparation in the layer 3 contains a silicone system which forms a soft and skin-friendly crosslinked elastomer, by means of a crosslinking reaction, after having been spread on the skin. The rate of this crosslinking reaction is already sufficient at the temperature which is imparted to the preparation on its contact with the skin, i.e. 20-40° C., and the material is in practice finally cured after 1 minute-24 hours. In this connection, “in practice finally cured” is understood as meaning that the material has reached a hardness which corresponds to a penetration value which is less than 2 mm greater than the value after the reaction has come to an end. The elastomer which is formed has a substantially higher cohesion than commercially available ointments/creams/pastes and, in addition, adheres to the skin in a skin-friendly manner, which means that the skin is not harmed when the preparation is removed.

The preparation layer 3, which is ointment-like on application, contains a silicone composition which, at 20-40° C., crosslinks spontaneously to form a soft elastomer. RTV silicone systems which are addition-curing and which can be crosslinked at room temperature are especially suitable. RTV silicones can be made soft and pliable. RTV stands for “room temperature vulcanizing”.

Examples of RTV addition-curing silicone systems are given in EP 0 300 620 A1, which describes what are termed “gel-forming compositions” which consist of an alkenyl-substituted polydiorganosiloxane, an organosiloxane containing hydrogen atoms linked to some of the silicone atoms, and also a platinum catalyst.

The chemical composition of RTV silicones is also described in U.S. Pat. No. 6, 471, 985 B2, which also describes a wound dressing which is produced in-situ.

Variants of these materials can be optimized for use as elastomer-forming leakage sealing on skin in accordance with this invention.

An example of a commercially available RTV silicone is Wacker SilGel 612 from Wacker-Chemie GmbH, Munich, Germany. This is a 2-component system. The softness of the elastomer which is formed can be varied by varying the proportions of the two components A:B from 1.0:0.7 to 1.0:1.3.

Examples of other soft silicone elastomers which adhere to dry skin are NuSil MED-6340, NuSil MED3-6300 and NuSil MED 12-6300 from NuSil Technology, Carpinteria, Ga., USA and Dow Corning 7-9800 from Dow Corning Corporation, Midland, USA.

The commercially available RTV silicones frequently also contain an inhibitor for the purpose of reducing the rate of reaction at low temperature, i.e. for the purpose of prolonging the time before the material cures spontaneously following admixture. In the application according to the invention, there is in certain cases a need for a more rapid crosslinking than that provided by most of the standard silicones of the addition-curing RTV type which are available on the market and which otherwise possess suitable properties. In such cases, it is possible to use the same grade but with a lower content of inhibitor than that present in the standard grades; alternatively, it is possible to leave out the inhibitor. It is also possible to substantially shorten the curing time by increasing the quality of catalyst 10-fold. PCT WO/73376 A1 describes the use of the inhibitor and catalyst for regulating the crosslinking reaction.

The preparation in the layer 3 can comprise a number of additives for different purposes, for example paraffin or ZnO for regulating the rheology, paraffin for reducing the adherence to skin, urea for reducing dehydration of the skin, antiinflammatory preparations, such as hydrocortisone, antimicrobial preparations, buffering components for promoting the skin healing process, agents, such as ZnO, for visualizing the ointment, etc. It is advantageous to use additives to make the preparation thixotropic since a thixotropic material is less viscous when it is being spread on the skin but becomes more viscous, and runs less, once it has come into position and the preparation is no longer being worked. The thixotropicity can be increased by adding silica and other fillers. There are also known methods for increasing the thixotropicity by adding silicone-based substances.

The viscosity of the preparation on application should be 5-300 Pa·s, preferably 10-120 Pa·s, more preferably 20-80 Pa·s, and the time until the ointment is in practice finally cured should be 0.5 min-24 hrs, preferably 1 min-1 hr, most preferably 1-5 min.

After curing, the ointment should have a penetration (softness) of 2-15 mm, preferably 3-10 mm, an adherence to skin after having cured against a Teflon plate which is less than 2.0 N/25 mm, preferably less than 1.0 N/25 mm, more preferably less than 0.7 N/25 mm, an adherence to skin after curing on skin of 0.3-3.0 N/25 mm and a skin damage index, Hx, which is less than 0.1, preferably less than 0.05.

Attention is drawn to the fact that the abovementioned adherence values relate to an ointment which is applied to dry and clean skin.

The crosslinking reaction affords a possibility of using the preparation to effectively attach other dressings over the region of a wound and a possibility of being able to remove the preparation in one piece. FIG. 2 shows a diagram of such an application, in which a dressing 4 has been applied over the wound 1 and attached to the preparation layer 3. The dressing 4 comprises a wound pad 5 composed of an absorbent material, a perforated layer 6 composed of a soft hydrophobic silicone adhesive, which does not become attached to the wound surface, and an outer, liquid-tight layer 7 which is composed, for example, of plastic material. Due to the fact that the preparation layer 3 has an ointment-like consistency, with a viscosity between 5-300 Pa·s on application to the skin around the wound, it will flow in to all the irregularities in the skin. The preparation layer 3 will consequently come to be in close adhesive contact with all parts of the skin around the region of the wound and thereby reliably prevent liquid from being able to pass between the layer 3 and the skin. The dressing 4 is preferably applied with its outer part covering the layer 3 before the layer 3 has cured to form an elastomer. This thereby ensures a close adhesive contact between the lower side of the perforated layer 6 and the upper side of the layer 3. Furthermore, the outer side 7 of the dressing 4 prevents exudate which has been sucked up from leaking out of the absorptive body. A design of this nature results in the wound bed being surrounded by a liquid-tight barrier on all sides. It is important that the layer 3 is applied such that the wound surface is kept free from the preparation in the layer 3 in order to prevent any absorption of exudate in an overlying dressing.

Examples of dressings which are provided with soft perforated layers of silicone adhesive are Mepilex, Mepilex Border, Mepilex Transfer and Mepitel from Mölnlycke Health Care AB, Gothenburg, Sweden.

Other types of dressing than the dressing 4 can naturally interact with the preparation of which the layer 3 is composed, for example traditional absorbent dressings having a surface which consists of a perforated plastic film, a nonwoven material or a textile material, for example dressings of the type Alldress, Mepore and Mesorb from Mölnlycke Health Care AB, Gothenburg, Sweden, or Melolin from Smith & Nephew Wound Management Ltd., Hull, Great Britain.

The preparation is applied to the skin and the article for medical use which it is intended to affix is placed in the desired position while the preparation is still a highly viscous liquid. After that, the preparation is allowed to crosslink. It may be expedient to select the material and surface structure of the article which is to be affixed such that the adherence of the preparation to the article is greater than to skin after the crosslinking reaction. In principle, the degree to which a material adheres to the preparation is directly proportional to the coarseness and raised nature of the surface structure of the material and to the magnitude of its contact area. The highest degree of adherence is obtained when use is made of a surface material for the article where the preparation has the possibility of forming bridges and lattices which enclose parts of the surface material. Examples of such materials are textile materials, nonwoven fabrics and foam having open pores. When the preparation is laid on the surfaces, it can penetrate into the material and enclose fabrics, or cell walls in the foam, such that a mechanical anchoring, by means of what are termed interpenetrating lattices, is obtained after curing. Account must also be taken of any substances which function as catalyst inhibitors and may be present in articles for medical use. When in contact with the preparation, these substances can entirely or partially prevent the crosslinking reaction. In many cases, it may be expedient to elaborate the preparation layer 3 such that its adherence to the layer 6 of the dressing is greater than to skin, with the layer 3 accompanying the dressing 4 when the latter is removed.

It is naturally also possible to first apply the preparation to the article for medical use and then to place the article, together with the applied preparation layer, in the intended position on the skin.

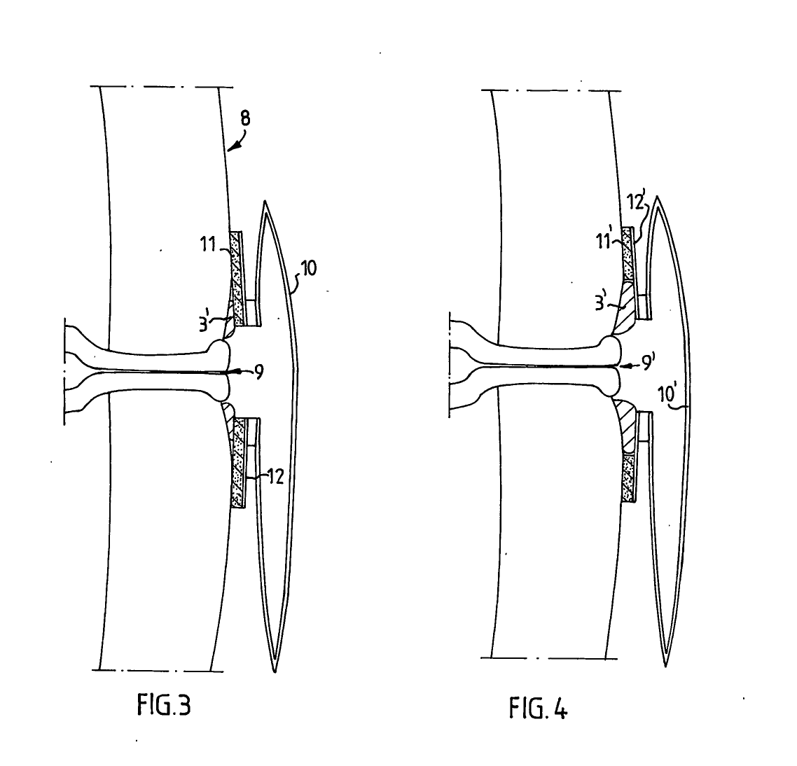

Apart from constituting a protective layer, the preparation can also function as a skin-friendly glue for gluing articles for medical use to the skin. FIG. 3 shows, in diagram form, how a layer 3′ of a preparation according to the invention is attached to the skin 8 around an intestinal opening 9. In addition, a stoma bag 10 is attached to the skin outside the layer 3′ using a glue layer 11 which is attached to a circular supporting plate 12. FIG. 4 shows a variant which only differs from the embodiment shown in FIG. 3 in that the glue layer 11′ of the stoma bag 10′ does not extend within the region of the protective layer 3′. Corresponding components in the two embodiments have been given the same reference numbers with a prime sign being added in the case of the components in FIG. 4.

An expedient method for supplying the preparation is in a two-chamber system series which is provided with a mixing nozzle. In this way, the two reactive silicone prepolymers can be kept separate and unreacted until the components are pressed out through the nozzle. This two-component addition-curing system can also be supplied ready-mixed. In this case, it is necessary to add a sufficient quality of inhibitor of the type which is described below. This completed mixture has a limited time for being used before it spontaneously crosslinks, and has to be stored cold in order to delay premature curing.

The preparation which has been described, and of which the layer 3 is composed, can be used on skin where ointments and creams are normally used for protection and treatment, for example skin around wounds (periwound skin), sensitive skin (not periwound), damaged skin/skin diseases (eczema, psoriasis, etc.) and skin which is subject to external disturbances (mechanical, chemical, water and microorganisms).

The above-described preparation layer 3 is a skin-protective product which can be provided with most of the positive properties possessed by ointments/pastes and barrier creams but which, at the same time, lacks important disadvantages of these latter. While the preparation can be used in all of the situations in which ointments/creams and pastes are normally used, it also has a wider use within other areas due to the novel properties, over and above the ointment/paste properties, which the preparation possesses, first and foremost the good adherence combined with a high degree of cohesion. The preparation also has major advantages as compared with the abovementioned volatile barrier products.
The preparation, which is ointment-like on application, adheres well to the skin due to its stickiness and protects the skin from wound liquid and other liquids. The hydrophobicity of the preparation contributes to keeping water and aqueous liquids away from the surface of the skin. A virtually complete contact is created between the skin and the preparation by the preparation being allowed to flow down in the skin before it solidifies. After the preparation has solidified and formed an elastomer, having a substantially higher cohesion than on application, a mechanical bonding to the skin also takes place since the preparation forms a precise impression of the skin (“key in keyhole”). No channels, where liquids would be able to run in and damage the skin, are formed in the interstice. The feature is skin-friendly and copes with body movements since the preparation is soft and flexible even after the crosslinking. The adherence is sufficiently high for reliable fixing without skin cells being stripped off when the preparation is removed. The strength of the adherence to the skin can be regulated by means of the choice of silicone composition or degree of binding or by means of adding quantities of, for example, ointment bases, silicone oil or ZnO.
The silicone-based preparation, which is ointment-like on application, attaches, after crosslinking, to many types of wound dressing (for example the majority of foam-based or fiber-based dressings) if the latter are laid on top of the preparation while the preparation still has an ointment or paste consistency. In connection with the crosslinking of the preparation, the latter binds to these overlying dressings by means of mechanical and adhesive binding. The mechanical binding results from the fact that part of the dressing (for example fibers, threads or cell walls in the foam) are enclosed by the silicone material. When the dressings are removed, the solidified preparation accompanies them. This facilitates handling and shortens the wound bandaging time. It is especially advantageous that the preparation also adheres to the silicone-coated surface of dressings of the Mepitel, Mepilex or Mepilex Border type (Mölnlycke Health Care, Gothenburg, Sweden). There are no known adhesives or plasters on the market which adhere as well to the soft silicone surface of the underside of the said products. When there is a requirement for the preparation to be pulled off in conjunction with a change of dressing, the preparation should be prepared such that the adherence to the skin is lower than the adherence to the dressing.
Since the preparation, which is ointment-like on application, functions as an adhesive towards the skin, on its lower side, and towards a dressing, on its upper side, when it has solidified, it is possible to produce a liquid-tight barrier by spreading the preparation on the area around the wound and laying a liquid-tight dressing, for example Mepilex Border or Mepiform (both from Mölnlycke Health Care AB, Gothenburg, Sweden) on top. The solidified ointment and the dressing together form a barrier which prevents liquids and contaminants (for example water, urine and feces) from penetrating into the wound from the exterior. At the same time, wound liquid is prevented from being able to emerge from the wound and contaminate the surrounding area. The wound liquid remains in the wound or is forced up into the dressing above, if this latter is an absorbent dressing.
Due to the fact that the preparation crosslinks spontaneously, it more readily stays put at the intended location. The risk of it being spread out on the skin outside the dressing or down into the wound decreases. Nor does the preparation disappear from the skin as does an ointment which is gradually able to migrate upward and be absorbed in an overlying dressing, where it can also block the intended absorption of wound liquid.
If the preparation is used in combination with a dressing which does not entrain it when the dressing is removed, it is instead possible to remove the dressing using a pair of forceps since the preparation remains coherent and can therefore be pulled off in one piece (or a few pieces). It is of course also possible to produce a gripping tab or the like when applying the preparation, for example by means of affixing a piece of release paper, or similar material to which the preparation does not adhere, to a part of the area of the skin to which the preparation is applied. The release paper can then be removed after curing and then leaves a part of the preparation which is not attached to the skin. In this case, this part of the preparation is advantageously applied such that it projects laterally from the remainder of the applied preparation. It is of course also possible to design the gripping tabs using a material which adheres to the preparation, by means of allowing a gripping part of such a material to project outside the preparation and only attaching an anchoring part of the material to the preparation.
Despite the fact that it adheres to the skin after the curing reaction, the preparation is very skin-friendly as compared with traditional adhesives. According to Dyke's method, it either causes no damage to the corneocyte layer or else less damage than do traditional adhesives. If the preparation is allowed to solidify to form a soft elastomer, hairs which grow on the skin beneath the crosslinked silicone preparation will not get caught and accompany the preparation when the latter is removed.
The preparation is also suitable for being used instead of hydrocolloid-based self-adhesive glue plates for fixing stoma bags (colostomy and ileostomy, where it is important to maintain leak-tightness and at the same time protect the sensitive skin.
The preparation is also suitable for producing leak-tightness when treating necrotic wounds with fly maggots, which are confined in the wound such that they are unable to escape, i.e. what is termed larvae treatment. In this connection, the preparation is spread immediately outside the wound where fly maggots have been placed, after which a close-meshed net, which does not allow the larvae to pass through, is laid over the wound and the ointment preparation, which is allowed to solidify. The preparation functions as an adhesive towards both the skin and the net.
The preparation can also be used for fixing other articles for medical use, for example tubes, etc., to the skin in a skin-friendly manner.
The ointment/elastomer is an excellent carrier for skin-protective substances which it is desired to supply to the skin, for example zinc oxide, pH buffer, petroleum jelly, urea and vitamins.

It is important that the preparation should be of the correct viscosity on application, before it becomes crosslinked. Different situations require different viscosities.

Different silicone-based preparations and commercially available ointments, pastes and creams were spread on the skin of experimental subjects, in layers of differing thickness and different sites on the body. The viscosities of the different preparations were measured using a CSL 100 Carri-Med Rheometer at a temperature of 30° C. and a shear rate of 10 s⁻¹. Results for some of the tested preparations, i.e. Wacker SilGel 612 (A:B=1:1, in the added presence of 50% ZnO), Vitt Vaselin [white petroleum jelly] (Apoteksbolaget Produktion och Laboratorier [The Pharmacy Company Production and Laboratories), Gothenburg, Sweden), Inotyol (Laboratoires Urgo [Urgo Laboratories), Dijon, France), Silon (Smith & Nephew, Mölndal, Sweden), ACO zinc paste [ACO hud AB [ACO Skin AB], Upplands Vasby, Sweden), Natusan zinc ointment (Johnson & Johnson Ltd., Maidenhead, SL6 3UG, UK) and EPSE Imprint II Garant Monophase (3M SPE Dental Products, St. Paul, Minn. 55144-1000, USA), can be seen in the following table:

| Preparation | Viscosity (Pa*s) |
|---|---|
| Inotyrol | 42 |
| Silon | 20 |
| Vitt Vaselin [white petroleum jelly] | 18 |
| zinc paste | 18 |
| zinc ointment | 24 |
| Imprint, light-green component | 74 |
| Imprint, dark-green component | 76 |
| SilGel 612 containing 50% ZnO | 14 |

It was possible to increase the viscosities of the preparations by adding fillers, for example ZnO or silica. Experiments to determine how different preparations functioned at different viscosities were also carried out by cooling the preparations to different temperatures. In one Wacker SilGel 612 (A:B=1:1) experiment, the viscosity could be increased appreciably by admixing ZnO (see table below). The viscosity values were read off 1 minute after mixing the components together.

Viscosity of Wacker SilGel 612 containing different
quantities of added ZnO (% by weight)

| | 0% ZnO | 25% ZnO | 35% ZnO | 50% ZnO | 65% ZnO |
|---|---|---|---|---|---|
| Viscosity | 0.7 | 1.7 | 2.9 | 14 | 51 |
| (Pa*s) | | | | | |

We found that preparations having viscosities within the interval 5-300 Pa·s worked well in different user situations. At even higher viscosity, the preparations become so viscous that they can no longer be used in this application. In this present document, highly viscous liquids are consequently understood as meaning liquids within the abovementioned interval. A preparation having a viscosity within the lower part of the interval may be easier to mix, for example in a static mixer, and may be easier to spread. However, there is then a higher risk of the preparation flowing out to too great an extent and not properly remaining at the intended site until it solidifies. This is particularly the case if the preparation is affected by movements of the body, pressure or friction. The higher the viscosity of the preparation, the easier it is for the preparation to be laid on in a thicker layer; at the same time, the preparation remains more reliably in place. On the other hand, preparations of high viscosity can be more difficult to mix and spread since they are relatively thick-bodied.

Preparations which, on application, have a viscosity within the interval 10-120 Pa·s function best. In most cases, preparations having a viscosity within the interval 20-80 Pa·s function best of all, especially if the need is to use the preparation to fix a dressing or other articles for medical use to the skin. Many skin ointments and skin pastes which can be purchased in a pharmacy have a viscosity precisely within this interval, with this facilitating application to the skin.

By means of adding fillers, it is also possible to give silicones thixotropic properties, with this being advantageous in connection with handling. Fumed silica, as is sold, for example, by Wacker Chemie under the trade mark Wacker HDK, was especially effective for this purpose.

Another important property of the preparation is its softness after the crosslinking has taken place. The softness is measured at penetration in mm units, by means of allowing a cone having a defined geometry and weight to sink down in the sample over a specified period of time. In the case of soft material, the cone will sink more deeply, resulting in a higher penetration value than otherwise obtained in the case of hard material, where the cone will not sink as deeply. The method is described in more detail in EP 0 782 457, to which document the reader is referred. Materials which are too hard can be insufficiently flexible for the user, especially if the material is lying in a relatively thick layer. Materials which are too soft can be troublesome to remove due to their stickiness and sometimes lower cohesion.

The softness of the crosslinked material is affected by a number of parameters, for example the degree of crosslinking and the admixing of fillers. In one experiment, an investigation was carried out to determine how the softness of the solidified silicone material is affected by admixing ZnO and by its degree of crosslinking.

We mixed the two components in Wacker SilGel 612 in different ratios and measured the penetration:

| A:B mix | Penetration (mm) |
|---|---|
| 100:80 | 13.2 |
| 100:90 | 15.8 |
| 100:100 | 20.4 |

When the ratio was decreased to 100:130, the penetration increased still further, just as it decreased still further when the ratio was increased to 100:70. The penetration values are to some extent batch-dependent, with it possibly having to be necessary to modify the A:B ratio in the case of each batch, in order to reach the desired penetration value.

A filler can be added for the purpose of increasing the hardness (decreasing the penetration value) of this material. When 50% ZnO was added to the 100:80 mixture, a penetration value of 7.3 mm was achieved. When the ZnO content was further increased to 60%, a penetration of 5.9 mm was achieved.

It was found that the preparation can function in said applications when it achieves a penetration value within the interval 2-15 mm after curing. Material having a penetration in the interval 3-10 mm functioned best.

It is well-known that it is possible to regulate the curing rate of addition-curing platinum-catalyzed RTV silicones by varying the quantity and type of catalyst and the quantity and type of inhibitor. The curing rate also naturally depends, inter alia, on the molecular weights, degree of branching and degree of substitution of the polymer components and on the quantity and type of crosslinker. All of this is well-known to professionals and is well described in literature.

Soft silicones are best suited to this invention. It was chosen to carry out experiments using SilGel 612 supplied by Wacker. A composition which gave a penetration value of around 5 mm in the added presence of 50% ZnO was chosen. This mixture had a curing time of about 4 hours at 30° C. A shorter curing time is required in some applications. On those occasions, it is possible to increase the quantity of catalyst. When an increased quantity of the manufacturer's original catalyst was added, the curing time was shortened. When a silicone system which was similar, but which lacked inhibitor in the system, was used instead, the curing time was reduced to less than 30 min. The curing time was determined by means of measuring penetration, with curing being regarded as having been achieved when the penetration value was less than 2 mm higher than the final value. The experiment demonstrates that it is simple to adjust the curing time to the desired level by modifying the quantities of catalyst and inhibitor. By means of allowing the silicone material to cure when it is in place on the skin, it is possible to produce an adherence to the skin which is appreciably more secure against leakage than if the same material had firstly been allowed to cure on the surface of a dressing and then applied to the skin. The following experiment supports this conclusion:

100 mm-long and 25 mm-wide strips of an inelastic textile material, grammage approx. 30 g/m², were coated with an approx. 2 mm-thick layer of Wacker SilGel 612 containing 50% by weight ZnO. Two different A:B ratios were chosen, i.e. 100:80 and 100:90. The textile material was impregnated right through and entirely covered on both sides by the silicone material. Half of the samples were allowed to lie and cure on a Teflon-coated heating plate at 30° C. The other half were placed on the skin of an experimental subject and allowed to cure. When the samples on the heating plate had cured, they were also placed on the skin of the experimental subject, alongside the other samples. After that, the samples were removed at a withdrawal angle of 135° and a speed of 25 mm/s, with the withdrawal force being read off concurrently. This method is also described in EP 0 782 457, to which the reader is referred.

| | Force of withdrawal | Force of withdrawal |
|---|---|---|
| | from skin after curing | from skin after curing |
| | in situ (N/25 mm) | on heating plate (N/25 mm) |
| Wacker SilGel | 0.3 | 0.1 |
| 612, 100:80 | | |
| Wacker SilGel | 2.9 | 0.8 |
| 612, 100:90 | | |

The measurement results show that the adherence of the preparation to the skin is appreciably greater when it has been applied uncured to the skin surface as compared with when it is cured before being applied. The preparations which function best exhibit at least twice the force of adhesion to skin when they are cured in situ.

An important property of the preparation is that, while it exhibits good adherence, and good liquid-sealing to skin after curing, this is without it being allowed to cause damage to the skin when it is removed. An experiment which illustrates this property is described below. The method which was used is a modification of a method which was published in Journal of Wound Care, Vol. 10, No. 2, 2001; Effects of Adhesive Dressings on the Stratum Corneum of the Skin, P. J. Dykes, R. Heggie and S. A Hill.

The inner sides of the forearms of an experimental subject were washed carefully by being rubbed with soap and water and then dried. The color of the skin was measured at the positions at which samples were subsequently to be applied (F₁). A color-measuring instrument, i.e. Minolta Chroma Meter, was used for measuring the color. The instrument was set to the color scale a*b*L, with the b value representing the green/red axis in the color scale. The greener the area under examination is, the lower is the b value. The less green, i.e. the more red, the area is, the higher is the b value.

A cotton wad was dipped into concentrated green foodstuff coloring. Manufacturer: Ekströms, Sweden. Content: water, glycerol, dyes E104 and E131, and ethanol. The cotton wad was stroked 20 times against the inner side of the forearm such that an approx. 3*20 cm-sized area in the longitudinal direction of the arm was colored green. After the color had been allowed to dry properly, the arm was rinsed under running water for approx. 1 minute at the same time as the colored skin was rubbed, uniformly over the entire area, with the inside of the other hand in order to remove the excess of color. The color of the skin was measured again at the same positions as before carrying out the green coloring (F₂).

Sample strips of 25*100 mm in size were applied such that they covered the positions where the color measurements had been made. The samples were applied transversely on the inner side of the forearm. The following samples were used:
a. inelastic but soft nylon textile material (approx. 30 g/m²) enclosed in an approx. 2 mm-thick layer of Wacker SilGel 612 containing 50% by weight of ZnO. A:B=100:80.
b. as sample a, but with an A:B=100:90.
c. Tielle, dressing for open wounds (Johnson & Johnson, USA). The self-adhesive edge was used in the experiment.
d. Mefix, self-adhesive acrylate glue-coated fabric for fixing dressings, etc., to skin (Mölnlycke Health Care AB, Sweden)
e. Duoderm, self-adhesive hydrocolloid dressing for treating open wounds (Convatec, USA)
f. Micropore, skin-friendly fixing tape (Johnson & Johnson, USA).
g. Symphony SimCare stoma dressing (Förbandsmaterial [dressing material] FMAB, Partille, Sweden). The self-adhesive surface which is used for fixing the stoma dressing around the intestinal aperture was employed.

After samples a-d had solidified fully, all the samples were removed at the same time as the withdrawal force was measured using the previously mentioned method. The samples were placed on a white substrate with the side which had been attached to the skin facing upward (U.S). The color was measured at two sites on the samples, i.e. on the one hand at that part which had lain on uncolored skin (F₃) and, on the other hand, at that part which had lain on colored skin (F₄).

A skin damage index was calculated for each sample, with this index being a measure of the quantity of the outer layer of the epidermis which accompanies the dressing when the latter is removed. The less the epidermis is damaged, the lower is the Hx. The skin damage index (Hx) was calculated using the formula:
Hx=(F₄−F₃)/(F₂=F₁)

Results:

| | Withdrawal force | Skin damage |
|---|---|---|
| Dressing or preparation | (N/25 mm) | index, Hx |
| Wacker SilGel 100:80 + | 0.3 | 0.09 |
| 50% ZnO (a) | | |
| Wacker SilGel 100:90 + | 2.9 | 0.01 |
| 50% ZnO (b) | | |
| Tielle (c) | 0.4 | 0.52 |
| Mefix (d) | 0.7 | 0.77 |
| Duoderm ET (e) | 3.4 | 1.00 |
| Micropore (f) | 0.1 | 0.38 |
| Symphony(g) | 0.2 | 0.87 |

The results show that, despite the silicones which cured in situ exhibiting a higher adhesive force, this did not result in any increase in the removal of cells from the epidermis.

Experimental subjects having little skin pigmentation were used in order to obtain the highest degree of precision when measuring the color.

The experiment can also be carried out using methylene blue as in the abovementioned published method (Dykes/Heggie/Hill). The blue/yellow axis in the a*b*L color scale is then used instead. The results are analogous.

One experiment was carried out by applying samples a and b from the above experiment to hair-covered skin before the silicone had cured. After curing, it was observed that only occasional hairs accompanied the samples when the latter were removed from the skin. This was a substantial difference as compared with samples of the Mefix, Duoderm and Tielle type which, on being removed, frequently take with them a large proportion of the hairs beneath the dressing.

1. A preparation for applying to the skin (stratum corneum), characterized in that it comprises a silicone composition which is highly viscous on application and which, after application, cures, by means of crosslinking, to form a soft and skin-friendly elastomer which adheres to the skin.

2. A preparation as claimed in claim 1, characterized in that, on application, it has a viscosity of 5-300 Pa·s, preferably 10-120 Pa·s, more preferably 20-80 Pa·s, and, after curing, has a penetration (softness) of 2-15 mm, preferably 3-10 mm.

3. A preparation as claimed in claim 1, characterized in that, after curing on the skin, it has an adherence to the skin of 0.3-3.0 N/25 mm.

4. A preparation as claimed in claims 1, characterized in that the curing time after application is 0.5 min-24 hrs, preferably 1 min-1 hr, more preferably 1-5 min.

5. A preparation as claimed in claims 1, characterized in that the preparation is hydrophobic.

6. A preparation as claimed in claim 1, characterized in that the silicone composition consists of an addition-curing RTV silicone system.

7. A preparation as claimed in claim 6, characterized in that the crosslinkable substance in the silicone system consists of polydimethylsiloxane having some of its methyl groups replaced with vinyl groups and the crosslinking-forming substance consists of dimethysiloxane having some of its methyl groups replaced with hydrogen, and a platinum-based catalyst.

8. A preparation as claimed in claim 6, characterized in that one or more skin-care substance(s) has/have been added to the silicone composition.

9. A method for applying a protective layer to the skin (stratum corneum), characterized in that a preparation comprising a silicone composition, which is highly viscous on application and which, after application, cures, by means of crosslinking, to form a soft and skin-friendly elastomer which adheres to the skin, is applied to the skin, after which the preparation is allowed to cure to form a soft, skin-friendly elastomer which adheres to the skin.

10. The method as claimed in claim 9, characterized in that the preparation is applied at a layer thickness of 0.1-5 mm.

11. The method as claimed in claim, characterized in that an article for medical use, such as a stoma bag, a tube or parts of a wound dressing or a bandage, is applied to the upper side of the preparation, i.e. that side which faces away from the skin, before the preparation has cured, with the article being affixed to the preparation after the latter has cured.

12. The method as claimed in claim 11, characterized in that the preparation is applied to the article for medical use before it is applied to the skin concurrently with the article.

13. The method as claimed in claim 11, characterized in that the preparation is designed such that its adherence to the article for medical use is greater than its adherence to the skin after curing.

14. The method as claimed in claim 9, characterized in that the preparation is applied around a wound, immediately outside the edge of the wound, with a width of 2-100 mm.

15. The method as claimed in claim 14, characterized in that one or more wound dressing(s) is/are applied such that the dressing(s) cover(s) the wound and the area to which the preparation has been applied, with the dressing(s) being applied before the preparation has cured.

16. The method as claimed in claim 15, characterized in that the wound dressing(s) consist(s) of (a) liquid-tight dressing(s).

17. A preparation as claimed in claim 2, characterized in that, after curing on the skin, it has an adherence to the skin of 0.3-3.0 N/25 mm.

18. A preparation as claimed in claim 7, characterized in that one or more skin-care substance(s) has/have been added to the silicone composition.

19. The method as claimed in claim 10, characterized in that an article for medical use, such as a stoma bag, a tube or parts of a wound dressing or a bandage, is applied to the upper side of the preparation, i.e. that side which faces away from the skin, before the preparation has cured, with the article being affixed to the preparation after the latter has cured.

20. The method as claimed in claim 12, characterized in that the preparation is designed such that its adherence to the article for medical use is greater than its adherence to the skin after curing.
